(19) United States
(12) Patent Application Publication
(10) Pub. No.: US 2021/0196615 A1
(43) Pub. Date: Jul. 1, 2021

(54) COSMETIC COMPOSITION FOR SKIN WHITENING

(71) Applicant: LG HOUSEHOLD & HEALTH CARE LTD., Seoul (KR)

(72) Inventors: Jun Seok YEOM, Daejeon (KR); Seong Cheon LEE, Daejeon (KR); Eu Gene HUR, Daejeon (KR); Yoon Kwan KIM, Daejeon (KR); Seol Hoon LEE, Daejeon (KR); Nae Gyu KANG, Daejeon (KR)

(73) Assignee: LG HOUSEHOLD & HEALTH CARE LTD., Seoul (KR)

(21) Appl. No.: 16/972,530

(22) PCT Filed: Jun. 7, 2018
(86) PCT No.: PCT/KR2018/006452

§ 371 Date: Dec. 4, 2020

(51) Int. Cl. A61K 8/88; A61K 8/11; A61Q 19/02; A61K 8/02; A61K 8/86; A61K 8/49; A61K 8/60; A61K 8/34; A61K 8/81; A61K 8/42; A61K 8/73; A61K 8/36; A61K 8/58; A61K 8/891; A61K 8/92

(52) U.S. Cl. CPC A61K 8/88; A61K 2800/651; A61Q 19/02; A61K 8/0212; A61K 8/86; A61K 8/496; A61K 8/60; A61K 8/345; A61K 8/8117; A61K 8/42; A61K 8/73; A61K 8/342; A61K 8/361; A61K 8/585; A61K 8/891; A61K 8/922; A61K 8/8111; A61K 8/11

(57) ABSTRACT

Disclosed is a cosmetic composition for skin whitening. The cosmetic composition for skin whitening contains a whitening component having a specific functional group capable of covalently bonding with a protein residue on the skin surface so as to form a covalent bond without damaging the skin, thereby semi-permanently providing a desired whitening effect.

### TECHNICAL FIELD

The present invention relates to a skin whitening component and a cosmetic composition for skin whitening.

### BACKGROUND ART

The whitening function of cosmetics refers to the function of making a skin color appear natural due to an active component as these cosmetics are continuously used to beautify the skin by brightening the color or tone of the human skin or covering skin flaws such as freckles or blemishes. This function blocks the expression of tyrosinase to prevent melanin formation or induces a mechanism of hindering the delivery of melanin, but to obtain whitening efficacy, such cosmetics need to be continuously used for a turn-over period (28 days of the period of skin keratin regeneration). In addition, by using a method of applying a cosmetic to the skin, it is difficult to obtain actual efficacy since a component without having affinity to the skin or hair is easily removed by washing or daily activities. Meanwhile, to obtain an instant effect, whitening cosmetics or color cosmetics, which are applied for external use, make the skin tone temporarily brighter, but are easily removed by washing or daily activities.

To improve the above-described disadvantages, there is a method of leaving or depositing a cosmetic component in the skin using various mechanisms such as a lipid having affinity to a surface of the skin or hair, a ceramide, a surfactant, a mineral imparting skin affinity, and a technique of increasing skin permeability, but an actual amount of the cosmetic component, which is left in the skin is very small, and the effect of the cosmetic component is also temporarily maintained. Accordingly, recently, a method of chemically modifying the surfaces of the skin and hair with a cosmetic material using a bioconjugation method has begun to be introduced. When this method is used to modify the stratum corneum and impart a beauty effect, the cosmetic effect may be maintained semi-permanently until desquamation, and if needed, the stratum corneum may be easily removed by peeling.

While L'Oreal has recently suggested a method of quickly obtaining an artificial tanning effect of dihydroxyacetone (DHA) by modifying the skin with an amine group-introduced alkoxysilane (US Unexamined Patent Application No. 2010-516546), due to high reactivity, the alkoxysilane is classified as Class 3 in MSDS toxicity, and thus is difficult to be applied in actual products. In previous research disclosed in US Unexamined Patent Publication No. 2011-0274639 and US Patent Publication No. 2006-0233729, methods of modifying skin were also proposed, but the technology of applying a bioconjugation method to cosmetics is in its early stage so that there are not many examples yet.

The present inventors have invented a method of modifying a skin protein with an amine group, a carboxyl group, a thiol group or a hydroxyl group, which was disclosed in previous research. Particularly, they have found a method of directly modifying proteins present in the hair and skin using polymeric carbodiimide (PCI) (Korean Patent Publication No. 2008-0064467) and are conducting research on applying this method in personal care. Since acyl urea is formed by a carbodiimide reaction, causing toxicity in the human body, such a reaction may be minimized by using a polymer-type carbodiimide. However, since reaction accelerators such as PCI, which can apply the corresponding reaction to the skin, are not diverse, to apply the bioconjugation method universally, there is a need for reaction media that are safe for the human body and can remain in the skin for a long time.

### DISCLOSURE

### Technical Problem

The present invention is directed to providing a cosmetic composition for skin whitening, which may effectively and continuously provide a semi-permanent whitening effect to the skin without damage.

### Technical Solution

To attain the above-described object, the present invention provides a cosmetic composition for skin whitening, which includes a peptide bond-forming accelerating additive in a carbodiimide composition.

### Advantageous Effects

A cosmetic composition for skin whitening according to the present invention can provide a skin whitening effect semi-permanently since a covalent bond is effectively formed in the skin by adding a reaction accelerating additive to a reactive skin whitening component to which a specific functional group enabling a covalent bond with a skin protein residue is introduced and/or a carbodiimide-based compound.

### DESCRIPTION OF DRAWINGS

FIG. 1 shows a result of a clinical test that quantifies the rate of improvement in skin brightness by 2-week use of a cream-type mask pack including glucosamine, a carbodiimide-based compound and a reaction accelerating additive (n=7, *p<0.05).

FIG. 2 shows a result of a clinical test that quantifies the rate of improvement in skin brightness by 1-week use of a cream-type mask pack including glucosamine, a carbodiimide-based compound and a reaction accelerating additive through a survey (n=20).

FIG. 3 shows a result of a clinical test that quantifies the rate of improvement in skin yellowness by 2-week use of a cream-type mask pack including chitosan, a carbodiimide-based compound and a reaction accelerating additive (n=7, *p<0.05).

FIG. 4 shows a result of a clinical test that quantifies the rate of improvement in skin gloss by 2-week use of a cream-type mask pack including chitosan, a carbodiimide-based compound and a reaction accelerating additive (n=7, *p<0.05).

### MODES OF THE INVENTION

The present invention relates to a cosmetic composition for skin whitening, which includes a carbodiimide-based compound, a reaction accelerating additive and a skin whitening component.

The cosmetic composition for skin whitening according to the present invention can be safely left on the surface of skin for a long time by forming a covalent bond with a skin protein by a reaction of the carbodiimide-based compound and the reaction accelerating additive with a conventionally known skin whitening component.

The carbodiimide-based compound is a generic term for compounds containing a structure represented by the following Formula 1 in a molecule, and is referred to as PCI in the present invention.

—N═C═N— [Formula 1]

The carbodiimide-based compound preferably includes at least approximately 1 to 1000 structures of Formula 1, more preferably include 1 to 100 structures represented by Formula 1 in the molecule, and still more preferably include 1 to 10 structures represented by Formula 1 in the molecule.

In this case, when there are more than 1000 carbodiimide groups represented by Formula 1 in one molecule, a raw material has a very high viscosity, a very large molecular weight and too many sites where the reaction occurs, and since a molecule containing carbodiimide groups becomes excessively larger, it hides a part exhibiting a functional effect of the molecule, which has a substantial function, causing reduced efficiency.

In the present invention, a specific example of the carbodiimide-based compound may be a compound represented by Formula 2 below, wherein the compound of Formula 2 in which n is 4 and m is 11 is referred to as benzene, 1,3-bis(1-isocyanato-l-methylethyl)-, homopolymer, polyethylene glycol mono-Me-ether-blocked (hereinafter, a polymer-type carbodiimide-based compound of Compound 2).

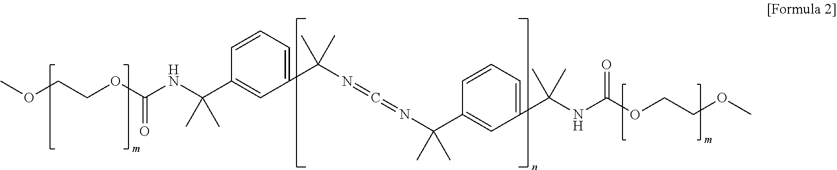

In Formula 2, n is 1 to 100, and m is 1 to 100.

The carbodiimide-based compound may be used at 0.001 to 10 parts by weight, 0.01 to 7 parts by weight or 0.1 to 5 parts by weight with respect to 100 parts by weight of the total composition. When the content is less than 0.001 parts by weight, it is difficult for a whitening component to sufficiently adhere to the skin surface, making it difficult to exhibit a continuous whitening effect, whereas when the content is more than 10 parts by weight, due to the excessive amount of the carbodiimide-based compound, which is more than reaction sites present on the skin surface, it is difficult to improve the efficacy, odor and properties of a product, and there are problems in formulation and formulation stabilization.

The “whitening effect” used herein refers to a function of beatifying the skin by brightening the skin by brightening the skin color or tone or improving the skin gloss or covering skin flaws such as freckles or blemishes appearing on the skin. In addition, the whitening effect also includes the function of adjusting a tone by preventing yellowness by preventing aging or oxidation on the skin surface.

The reaction accelerating additive for forming a peptide bond may be used at 0.0001 to 10 parts by weight, preferably 0.001 to 1 part by weight, and more preferably, 0.001 to 0.1 parts by weight with respect to with respect to 100 parts by weight of the total composition. When the additive is used at more than 10 parts by weight, since a chemical reaction speed is saturated, a product is not only uneconomical, but also not improved in stability. When the additive is used at less than 0.0001, it is difficult to exhibit an effect of adhering a whitening component onto the skin surface.

The reaction accelerating additive for forming a peptide bond may be, specifically, one or more selected from the group consisting of hydroxysuccinimide, hydroxycarbamide, hydroxytriazole, hydroxybenzotriazole, hydroxypyridinone, hydroxyphenylbenzimidazole, hydroxyindolinone, hydroxybenzotriazine and oxyma, but the present invention is not limited thereto. In addition, the reaction accelerating additive may be a water-soluble nonionic polymer, a bead or resin-type polymer resin, or combined with silica beads to increase stability and biosafety in the formulation.

The water-soluble nonionic polymer may have a weight average molecular weight of 1,000 Da or more and less than 300,000 Da, and when the molecular weight is 300,000 Da or more, it is difficult to exhibit efficacy because the proportion of the carbodiimide-based compound inducing a covalent bond is too small.

The water-soluble nonionic polymer may be, specifically, one or more polymers selected from the group consisting of polyethylene glycol, polyvinyl alcohol, polyethylene oxide, polyacrylonitrile, polyvinylpyrrolidone, polyisopropylacrylamide, a cellulose derivative, a starch derivative, dextran and guar gum.

It is preferable that the resin-type polymer has a weight average molecular weight of 1,000 Da or more and less than 300,000 Da, and when a molecular weight is 300,000 Da or more, it is difficult to exhibit efficacy because there is a very small proportion of the carbodiimide-based compound inducing a covalent bond.

When the bead-type polymer resin (polymer bead) may have an average particle size of 100 nm to 1 mm, preferably 100 nm to 100 μm, more preferably, 1 to 100 μm, and most preferably, 1 to 70 μm. When the polymer bead has a diameter of less than 100 nm, there may be a problem in terms of safety for the skin surface, and when the polymer bead has a diameter of more than 1 mm, there are problems in terms of formulation and stabilization.

The bead- or resin-type polymer resin may be a copolymer polymerized with a monomer selected from the group consisting of, specifically, styrene, ethylene, butadiene, acrylonitrile, methyl styrene, terephthalate, ethylene chloride, ether ketone, ether imide, ether sulfone, phthalamide, phenylene ether, phenylene oxide, phenylene sulfide, sulfone, urethane, vinylidene fluoride and tetrafluoroethylene.

The silica bead may have an average particle size of 100 nm to 1 mm, preferably, 100 nm to 100 μm, more preferably, 1 to 100 μm, and most preferably 1 to 70 μm. When the diameter of the silica bead is smaller than 100 nm, there is a safety problem on the skin surface, and when the diameter of the silica bead is larger than 1 mm, there are problems in terms of formulation and stabilization.

The skin whitening component used herein may contain a whitening component that can improve a region of the skin damaged by melanin pigmentation, such as extracts derived from natural substances such as animals, plants and minerals, amino acids, hormones, and vitamins, but the present invention is not limited thereto. For example, the component may be one or more selected from the group consisting of hydroquinone-based compounds; vitamins such as niacinamide and vitamin C and a derivative thereof; flavonoid compounds such as moistin and hesperidin; resveratrol compounds and a derivative thereof; curcubitacin derivatives; amino acid-based compounds such as glutathione and methylundecenoylucinate; arbutin, kojic acid, azelaic acid, alpha hydroxy acid, alpha-bisabolol, adenosine, ascorbyl glucoside, ascorbyl tetraisopalmitate, ethylascorbyl ether, fullerene, acetyl tyrosine, diacetyl benzoyl lathyrol, Atractylodes japonica root oil, magnesium ascorbyl phosphate, diosmethine, macelignan, acetyl sphingosine, dihydroxymethoxychalcone, alpha-bisabolol, protocatechuicaldehyde, oryzanol, a licorice extract, a paper mulberry extract, a green tea extract, a morus bark extract, an adlay extract, a yeast extract, an Atractylodes japonica extract, a wheat germ extract, a Paeonia lactiflora extract, a Lithospermum erythrorhizon root extract, a ginseng extract, a red ginseng extract, a Saururus chinensis extract, an Agrimonia eupatoria extract, an Canna indica root extract, a Sasa quelpaertensis extract, a Sophora flavescens root extract, a Coix lacryma-jobi ma-yuen seed extract, a Prunus domestica fruit extract, chitosan, polylysine, glucosamine and a derivative thereof, but the present invention is not limited thereto.

The skin whitening component may be used at 0.001 to 50 parts by weight, 0.001 to 30 parts by weight, 0.01 to 10 parts by weight or 0.1 to 5 parts by weight with respect to 100 parts by weight of the total cosmetic composition. When the content is less than 0.001, its effect is insignificant, and when the content is more than 50 parts by weight, there are problems in terms of formulation and stabilization.

Particularly, in the present invention, the skin whitening component may include a carboxyl group or amine group in the molecule, thereby improving reaction efficiency with the carbodiimide-based compound.

In addition, the skin whitening component of the present invention may have a functional group which makes a covalent bond with a protein residue of the skin surface.

The functional group which makes a covalent bond with a protein residue may be one or more selected from the group consisting of carbodiimide, imidoester, arylazide, diazirine, hydroxymethyl phosphine, pentafluorophenylester, pyridyl disulfide, sulfo-hydroxysuccinimide ester, alkoxyamine, hydrazide, haloacetyl, azide, carbonate, aldehyde, propionaldehyde, butylaldehyde, nitrophenyl carbonate, aziridine, isocyanate, thiocyanate, epoxide, trecylate, succinimide, hydroxysuccinimidyl ester, imidazole, oxycarbonyl imidazole, imine, thiol, maleimide, vinylsulfone, ethyleneimide, thioether, acrylonitrile, acrylic or methacrylic ester, disulfide, ketone, carboxylic acids and phosphate, but the present invention is not limited thereto.

In addition, the protein may include a reactive residue such as thiol, hydroxyl, carboxyl or amine.

As an exemplary embodiment of the cosmetic composition for skin whitening according to the present invention, the effect of the cosmetic composition may be significantly improved by forming a whitening component with a carboxyl group in the form of a reactive ester whitening component directly having bioreactivity, or improving reaction efficiency by forming an amino acid (e.g, aspartic acid or glutamic acid) excessively present in skin keratin (15.5 to 23.5%) in the form that can be targeted by the skin whitening component containing an amine group through reactive esterification. As a more preferable exemplary embodiment, an excellent whitening effect may be obtained by first forming a reactive ester by primary reaction of a polymer-type carbodiimide-based compound on the surface of a skin whitening component molecule or skin protein having a carboxyl group, substituting the reactive ester with the reaction accelerating additive to increase selectivity, and reacting a whitening component having amine in the molecule with the formed reactive ester functional group or amine on the skin surface.

The following Reaction Scheme 1 shows a schematic diagram of a reaction of forming a reactive ester by a primary reaction between a skin whitening component molecule having a carboxyl group and a carbodiimide-based compound, substituting the reactive ester with a reaction accelerating additive to increase selectivity, and forming a covalent bond with a biological amino acid having an amine group.

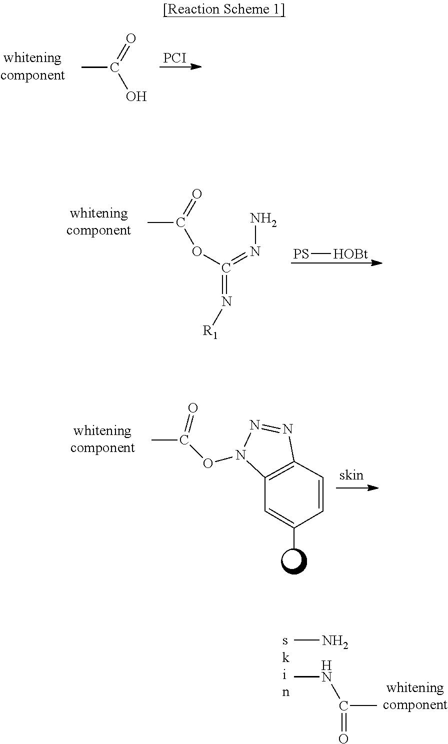

The following Reaction Scheme 2 shows a schematic diagram of a reaction of forming a reactive ester by the primary reaction between a biological amino acid with a carboxyl group, such as aspartic acid or glutamic acid, present on the surface of the skin protein, and a carbodiimide-based compound, increasing selectivity by substituting the reaction accelerating additive with the reactive ester, and forming a covalent bond by the reaction with a whitening component with an amine residue.

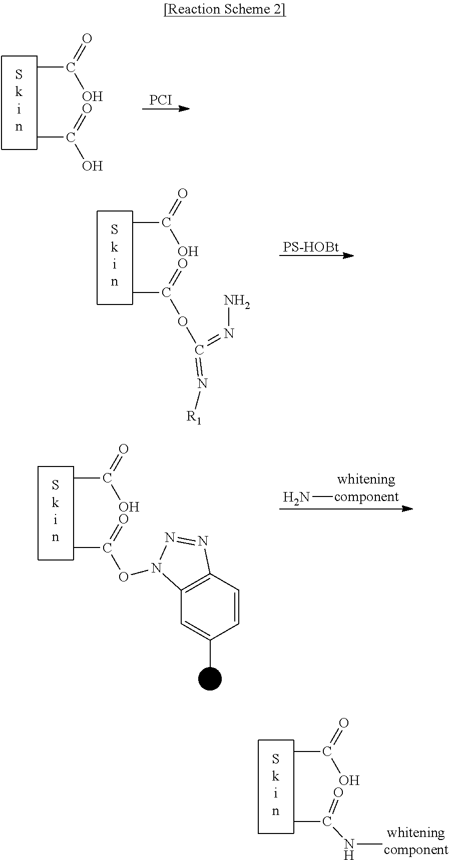

A skin whitening component adhered to a skin protein by peptide bonding using a carbodiimide-based compound may be adhered to the skin semi-permanently without removal from the skin by a washing method with a shampoo, soap or a cleanser.

Due to the improved reaction efficiency between the skin protein and the skin whitening component by adding the reaction accelerating additive to the carbodiimide-based compound, the whitening component adhered to the skin may not be easily dissociated in general washing with soap and may be almost permanently adhered to the skin.

This Reaction Scheme is a representative example for the skin and can also be applied to hair, skin, fingernails, toenails, fiber or leather.

The present invention may also include a reactive whitening component in which a functional group forming covalent bonds with one or more proteins selected from the group consisting of carbodiimide, imidoester, arylazide, diazirine, hydroxymethyl phosphine, pentafluorophenylester, pyridyl disulfide, sulfo-hydroxysuccinimide ester, alkoxyamine, hydrazide, haloacetyl, azide, carbonate, aldehyde, propionaldehyde, butylaldehyde, nitrophenyl carbonate, aziridine, isocyanate, thiocyanate, epoxide, trecylate, succinimide, hydroxysuccinimidyl ester, imidazole, oxycarbonyl imidazole, imine, thiol, maleimide, vinylsulfone, ethyleneimide, thioether, acrylonitrile, acrylic or methacrylic ester, disulfide, ketone, carboxylic acid and phosphate are bound to the skin whitening component.

For example, as shown in Reaction Scheme 1, a whitening component prepared by a primary reaction of a carbodiimide-based compound and then substitution with a reaction accelerating additive may be included. In addition, the present invention may include a cosmetic composition for skin whitening, which includes a whitening component substituted with the reaction accelerating additive.

In addition, by mixing with a fatty acid such as palmitic acid or stearic acid, a fatty alcohol, a cationic surfactant such as a linear or branched long-chain alkyl quaternary ammonium salt, a cationized polymer such as cationized cellulose, cationized guar gum or cationized polyvinylpyrrolidone, or silicone in order to additionally improve its effect, the cosmetic composition for skin whitening according to the present invention may be easily prepared. In addition, for formulation as a cosmetic preparation, components for cosmetic preparation, such as a solvent, a surfactant, a thickening agent, a stabilizer, a preservative, a colorant, a pH adjuster, a metal ion sequestering agent, a pearling agent, an appearance improving agent, an emollient, a pigment, powder particles, and a particle, may be additionally included. The component for preparation may be used at 40 to 99 parts by weight or 60 to 99 parts by weight with respect to 100 parts by weight of the total composition.

The cosmetic composition for skin whitening according to the present invention may include a skin whitening component having a functional group and thus may be used in formulation of the cosmetic composition for skin whitening. Examples of skin products may include, generally, skin care products for smoothing and protecting skin (toner, serum, essence, lotion, cream, etc.), color cosmetics (make-up base, foundation, powder, eye shadow, lip gloss, lipstick, lip balm, etc.), cleansing cosmetics (foam cleanser, cleansing oil, cleansing lotion, cleansing cream, cleansing gel, pack, mask, soap, cleansing tissue, etc.), sun care cosmetics, and body cosmetics (body lotion, shower gel, body cream, body oil, etc.).

More preferably, the cosmetic composition for skin whitening according to the present invention includes a whitening component having a reactive functional group as well as a carbodiimide-based compound and a reaction accelerating additive. If the above-described preparation has lowered activity in an aqueous state, it is easier to maintain the activity of the preparation in a non-aqueous formulation, and a reaction may be induced by mixing with a buffer to adjust a pH immediately before use or contact with water during washing. Examples of non-aqueous formulations may include liquid-, sheet-, powder- and tablet-type formulations, oil, ampoules, and gel, which have been used as conventional non-aqueous cosmetic preparations. In addition, the cosmetic composition for skin whitening according to the present invention may be formulated with a carbodiimide-based compound, a reaction accelerating additive and a whitening component as an encapsulated single agent or as two separated agents. In addition, a method of blocking a derivative in the form in which a reactive functional group and a component having a whitening function are bound from moisture through encapsulation may also be used.

To improve a whitening effect of the cosmetic composition for skin whitening according to the present invention with a functional group, a dibasic acid ester, such as dioctyl succinate, dioctyl adipate or diethyl sebacate, a polyol, polyethylene glycol, propylene glycol, hexylglycol, butanediol and an isomer thereof, hexylene glycol, butanediol and an isomer thereof, glycerol, benzyl alcohol, and ethoxydiglycol and a derivative thereof may be used. The above-described solvent increases the permeability of hair or skin, and is used as a solvent for an insoluble material. More preferably, solvents that are used to improve the activity retaining effect of a whitening component having a functional group include diethyl sebacate, ethoxyglycol, and bis-ethoxyglycol cyclohexane 1,4-dicarboxylate.

### EXAMPLES

Hereinafter, the present invention will be described in detail with reference to the following examples. The following examples are merely provided to exemplify the present invention, and the contents of the present invention are not limited to the following examples.

### Example 1 and Comparative Examples 1 and 2: Preparation of Cream (100 g)-Type Mask Pack

Cream-type mask packs were prepared with the compositions and contents shown in Table 1 below.

Water-phase components 3 to 13 were heated to 70° C., and then uniformly mixed for 5 minutes at 1500 rpm using a homomixer. Oil-phase components 15 to 24, which had been previously heated to 70° C., were added to the mixture of the water-phase components, and then uniformly mixed for 5 minutes at 3500 rpm using a homomixer. Subsequently, the temperature was decreased to 50° C., a counteractive, a carbodiimide-based compound such as benzene, 1,3-bis(1-isocyanato-1-methylethyl)-, homopolymer, polyethylene glycol mono-Me-ether-blocked (1), PS-HOBt (2) as a peptide bond-forming accelerating additive (reaction accelerating additive) and a fragrance (14) were added and then uniformly mixed for 3 minutes using a homomixer, followed by defoaming, filtration and cooling.

The hydroxybenzotriazole (PS-HOBt) was prepared by coupling a 10-μm bead-type polymer resin with hydroxybenzotriazole, wherein the polymer resin is in a form of 10-μm bead consisting of polystyrene represented by the following Formula 3 (molar ratio of m+n+x+y:z=99:1). The repeating unit in z below is a crosslinker which is not adhered to a specific location and randomly connects polymers, and synthesized with reference to Tetrahedron Letters 41 (2000) 2463-2466 and/or Chem. Rev. 2011, 111, 6557-6602.

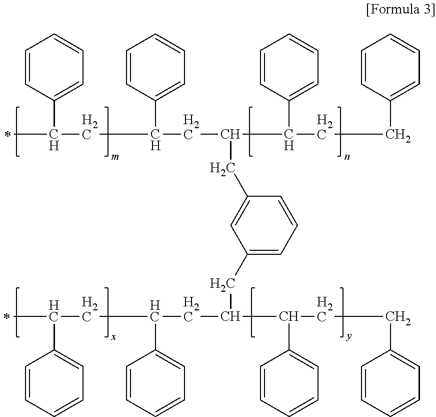

TABLE 1

| | | | Comparative | Comparative |
|---|---|---|---|---|
| No | Component | Example 1 | Example 1 | Example 2 |
| 1 | Benzene, 1,3-bis(1- | 0.1 | — | 0.1 |
| | isocyanato-1-methylethyl)-, | | | |
| | homopolymer, polyethylene | | | |
| | glycol mono-Me-ether- | | | |
| | blocked | | | |
| 2 | PS-HOBt | 0.1 | 0.1 | — |
| 3 | Glucosamine | 1.0 | 1.0 | 1.0 |
| 4 | Glycerin | 6.0 | 6.0 | 6.0 |
| 5 | DPG | 3.0 | 3.0 | 3.0 |
| 6 | 1,3-butylene glycol | 2.0 | 2.0 | 2.0 |
| 7 | Urea/HEPES | 0.8 | 0.8 | 0.8 |
| 8 | Preservative | 1.0 | 1.0 | 1.0 |
| 9 | Anti-inflammatory agent | 2.0 | 2.0 | 2.0 |
| 10 | Carbomer | 0.2 | 0.2 | 0.2 |
| 11 | Xanthan gum | 0.05 | 0.05 | 0.05 |
| 12 | Counteractive | 0.28 | 0.28 | 0.28 |
| 13 | Distilled water | To 100 | To 100 | To 100 |
| 14 | Fragrance | 0.1 | 0.1 | 0.1 |
| 15 | Cetearyl alcohol | 2.5 | 2.5 | 2.5 |
| 16 | Glyceryl stearate | 1.2 | 1.2 | 1.2 |
| 17 | PEG-100 stearate | 0.3 | 0.3 | 0.3 |
| 18 | PEG-40 stearate | 0.5 | 0.5 | 0.5 |
| 19 | Stearic acid | 0.3 | 0.3 | 0.3 |
| 20 | Cyclohexasiloxane | 6.0 | 6.0 | 6.0 |
| 21 | Petaerythrityl | 3.0 | 3.0 | 3.0 |
| | tetraethylhexanoate | | | |
| 22 | Dimethicone | 5.0 | 5.0 | 5.0 |
| 23 | Macadamia oil | 2.0 | 2.0 | 2.0 |
| 24 | Hydrogenated polydecene | 4.0 | 4.0 | 4.0 |

### Experimental Example 1

Glucosamine was adhered to the skin by bioconjugation using a carbodiimide-based compound and a reaction accelerating additive, and then its efficacy was confirmed.

Each of the mask pack formulations of Comparative Example 2 and Example 1 was prepared, and then applied to 7 subjects to confirm changes in skin brightness. The use of a mask pack was performed by cleansing a face with a cleanser before use, adhering the mask pack to the face for 30 minutes, and then washing the face again, the process of which was performed twice a day for 2 weeks. The skin brightness was checked with a LAB meter (Konica Minolta CR-300 Chroma Meter).

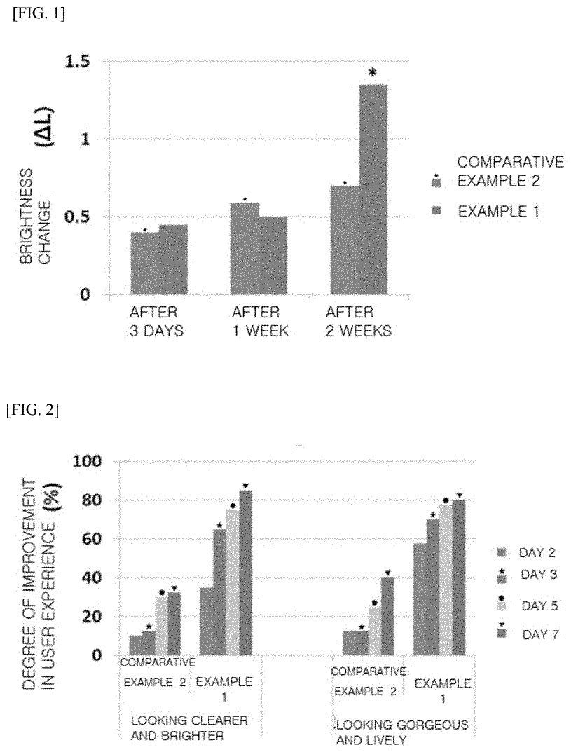

As a result of the experiment, when glucosamine was adhered using the carbodiimide-based compound and the reaction accelerating additive (Example 1), it was confirmed that the skin brightness was improved by 1.35% for 2 weeks [FIG. 1]. Compared with Comparative Example 2 using only a carbodiimide-based compound, the whitening effect in Example 1 was remarkably improved.

It is expected that not only glucosamine but also various whitening components are adhered to the skin with a reaction accelerating additive through bioconjugation remain for a long time, thereby continuously obtaining a whitening effect for a certain period.

### Experimental Example 2

Glucosamine was adhered to the skin by bioconjugation using a carbodiimide-based compound and a reaction accelerating additive, and then its efficacy was confirmed.

Each of the mask pack formulations of Comparative Example 2 and Example 1 was prepared, and then applied to 20 subjects to confirm changes in skin brightness. The use of a mask pack was performed by cleansing a face with a cleanser before use, adhering the mask pack to the face for 30 minutes, and then washing the face again, the process of which was performed once a day for 1 week.

The changes in skin brightness were evaluated using user questionnaires. By setting items such as a moisturizing property, a whitening property and elasticity, the degree of improvement in user's experience was confirmed as x when these properties are similar to usual or o when these properties seem to be improved.

As a result of the experiment, when glucosamine was adhered to the skin by bioconjugation using the carbodiimide-based compound and the reaction accelerating additive, it was confirmed that the skin brightness that had been felt by a user was improved by 80% or more for 1 week [FIG. 2]. Compared with Comparative Example 2 using only a carbodiimide-based compound, the whitening effect in Example 1 was remarkably improved.

It is expected that not only glucosamine but also various whitening components are adhered to the skin with a reaction accelerating additive through bioconjugation remain for a long time, thereby continuously obtaining a whitening effect for a certain period.

### Example 2

A mask pack was prepared in the same manner as in Example 1, except that chitosan was used instead of glucosamine used in Example 1.

### Comparative Example 3

A mask pack was prepared in the same manner as in Comparative Example 2, except that chitosan was used instead of glucosamine used in Comparative Example 2.

### Experimental Example 3

Chitosan was adhered to the skin through bioconjugation using a carbodiimide-based compound and a reaction accelerating additive, and then its efficacy was confirmed (the skin whitening effect was confirmed by adjusting a tone by reducing yellowness by preventing aging or oxidation on the skin surface).

Each of mask pack formulations of Example 2 and Comparative Example 3 was prepared, and applied to the cheeks of 7 subjects to confirm changes in skin yellowness and gloss, which are criteria of skin aging. The use of the mask pack was performed by cleansing a face with a cleanser before use, adhering the mask pack to the face for 30 minutes, and washing the face again, the process of which was performed twice a day for 2 weeks. Yellowness was confirmed using a LAB meter (Konica Minolta CR-300 Chroma Meter), and a change in gloss was confirmed using a glossmeter (C&K MPA 580, skin glossmeter GL200).

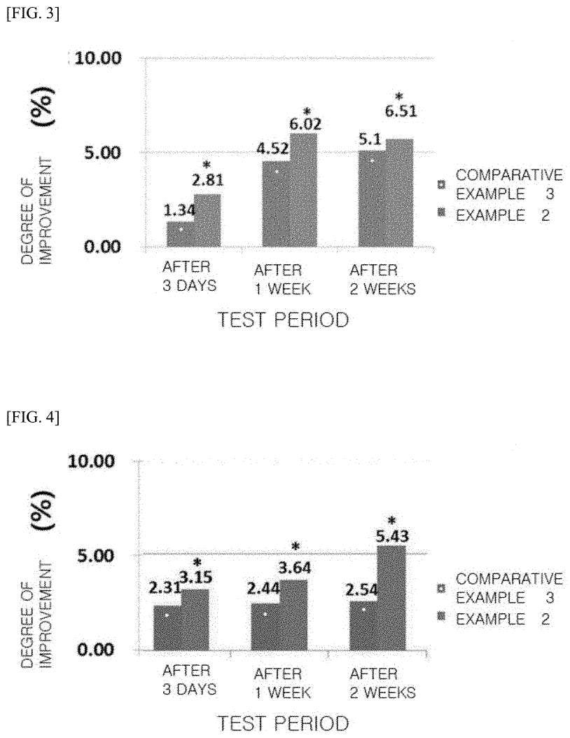

As a result of the experiment, when chitosan was adhered by bioconjugation with the carbodiimide-based compound and the reaction accelerating additive (Example 2), due to the antioxidation and antiaging ability of the chitosan, it was confirmed that skin yellowness was improved by 6.51% after two weeks of use, and skin gloss was also improved by 5.43% after two weeks of use [FIGS. 3 and 4]. Compared with Comparative Example 3 using only a carbodiimide-based compound, the whitening effect in Example 2 using the carbodiimide-based compound and the reaction accelerating additive was quickly and remarkably improved.

It is expected that not only chitosan but also various whitening components are adhered to the skin by bioconjugation remain for a long time, thereby continuously obtaining a cosmetic effect for a certain period.

### Example 3 and Comparative Examples 4 to 6: Preparation of Non-Woven Fabric Mask Pack

Non-woven type mask pack formulations were prepared with the compositions and contents of Table 2 below.

In further detail, water-phase components 1, 8, 9, 10, 11, 14, 15 and 16 were heated to 70° C., and uniformly mixed for 5 minutes at 1500 rpm using a homomixer. Oil-phase components 5, 6, 7, 12 and 13 which had been previously heated to 70° C. to dissolve, were added to the mixture of the water-phase components and then uniformly mixed for 5 minutes at 2000 rpm using a homomixer. Subsequently, the temperature was lowered to 50° C., benzene, 1,3,-bis(1-isocyanato-1-methylethyl)-, homopolymer, polyethylene glycol mono-Me-ether-blocked (2), a reaction accelerating additive (3) and glucosamine (4) and a fragrance (17) were added and uniformly mixed for 3 minutes using a homomixer, followed by defoaming, filtration and cooling. Afterward, a non-woven mask was impregnated with the resulting formulation.

TABLE 2

| | | Glucosamine (2%) | | | |
|---|---|---|---|---|---|
| | | | Comparative | Comparative | Comparative |
| No | Component (wt %) | Example 3 | Example 4 | Example 5 | Example 6 |
| 1 | Distilled water | To 100 | To 100 | To 100 | To 100 |
| 2 | Benzene, 1,3-bis(1-isocyanato-1- | 0.1 | — | — | 0.1 |
| | methylethyl)-, homopolymer, | | | | |
| | polyethylene glycol mono-Me- | | | | |
| | ether-blocked | | | | |
| 3 | PS-HOBt | 0.1 | — | 0.1 | — |
| 4 | Glucosamine | 1.0 | 1.0 | 1.0 | 1.0 |
| 5 | Dimethicone | 4.0 | 4.0 | 4.0 | 4.0 |
| 6 | Pentaerythrityl | 3.0 | 3.0 | 3.0 | 3.0 |
| | tetraethylhexanoate | | | | |
| 7 | Hydrogenated | 1.0 | 1.0 | 1.0 | 1.0 |
| | polydecene | | | | |
| 8 | Sorbitol | 3.0 | 3.0 | 3.0 | 3.0 |
| 9 | DPG | 7.0 | 7.0 | 7.0 | 7.0 |
| 10 | 1,3-butylene glycol | 1.0 | 1.0 | 1.0 | 1.0 |
| 11 | Glycerin | 7.0 | 7.0 | 7.0 | 7.0 |
| 12 | Bis-PEG-18 | 2.0 | 2.0 | 2.0 | 2.0 |
| | Methyl ether | | | | |
| | dimethyl silane | | | | |
| 13 | Methyl glucose | 0.3 | 0.3 | 0.3 | 0.3 |
| | sesquistearate | | | | |
| 14 | Preservative | 1.0 | 1.0 | 1.0 | 1.0 |
| 15 | Anti-inflammatory agent | 2.0 | 2.0 | 2.0 | 2.0 |
| 16 | Hydroxyethylcellulose | 0.4 | 0.4 | 0.4 | 0.4 |
| 17 | Fragrance | 0.1 | 0.1 | 0.1 | 0.1 |

### Experimental Example 4: Evaluation of Improvement in Skin Brightness

Glucosamine was adhered to the skin through bioconjugation between a carbodiimide-based compound and a reaction accelerating additive, and its efficacy was confirmed. Mask pack formulations of Example 3 and Comparative Examples 4 to 6 were prepared, and applied to 7 subjects to confirm changes in skin brightness.

The use of a mask pack was performed by cleansing a face with a cleanser before use, adhering a mask pack to the face for 30 minutes, and then washing the face again, the process of which was performed once a day for 2 weeks. The skin brightness was confirmed using a LAB meter (Konica Minolta CR-300 Chroma Meter).

Subsequently, after each test material was applied, the difference (ΔL*) in skin color between a measurement time and an application starting time was calculated by the following Equation 1, and the result is shown in Table 3 below.

Meanwhile, a whitening effect was determined by comparing ΔL* at the sample-applied site and a control site, and when the ΔL* value is approximately 2, the whitening of the deposited pigment is apparently shown, and when the ΔL* value is approximately 1.5 or more, it can be determined that there is a whitening effect.

ΔL*=L value at the measured time after application−L value at the application starting time [Equation 1]

TABLE 3

| | Glucosamine (2%) | | | |
|---|---|---|---|---|
| | | Comparative | Comparative | Comparative |
| | Example 3 | Example 4 | Example 5 | Example 6 |
| Day 2 | 1.3 | 0.6 | 0.7 | 0.9 |
| Day 7 | 1.6 | 0.8 | 0.8 | 1.2 |
| Day 14 | 2.3 | 0.9 | 0.7 | 1.8 |

As shown in Table 3, in terms of the whitening effect of glucosamine through the bioconjugation between the carbodiimide-based compound and the reaction accelerating additive, it was confirmed that, when the carbodiimide-based compound and the reaction accelerating additive were used, the skin brightness is remarkably improved for 2 weeks. It is expected that not only glucosamine but also various whitening materials are adhered to the skin through bioconjugation to remain for a long time, thereby continuously obtaining a cosmetic effect for a certain period.

1. A skin whitening composition comprising a carbodiimide-based compound, a reaction accelerating additive and a skin whitening component.

2. The skin whitening composition of claim 1, wherein the reaction accelerating additive is one or more selected from the group consisting of hydroxysuccinimide, hydroxycarbamide, hydroxytriazole, hydroxybenzotriazole, hydroxypyridinone, hydroxyphenylbenzimidazole, hydroxyindolinone, hydroxybenzotriazine and oxyma.

3. The skin whitening composition of claim 1, wherein the reaction accelerating additive is a water-soluble nonionic polymer, a bead-or resin-type polymer resin, or a type bound with silica beads.

4. The skin whitening composition of claim 3, wherein the water-soluble nonionic polymer has a weight average molecular weight of 1,000 Da or more and less than 300,000 Da.

5. The skin whitening composition of claim 3, wherein the water-soluble nonionic polymer is one or more polymers selected from the group consisting of polyethylene glycol, polyvinyl alcohol, polyethylene oxide, polyacrylonitrile, polyvinylpyrrolidone, polyisopropylacrylamide, a cellulose derivative, a starch derivative, dextran and guar gum.

6. The skin whitening composition of claim 3, wherein the resin-type polymer has a weight average molecular weight of 1,000 Da or more and less than 300,000 Da.

7. The skin whitening composition of claim 3, wherein the bead-type polymer resin has an average particle size of 100 nm to 1 mm.

8. The skin whitening composition of claim 3, wherein the bead- or resin-type polymer resin is a polymer polymerized with a monomer selected from the group consisting of styrene, ethylene, butadiene, acrylonitrile, methyl styrene, terephthalate, ethylene chloride, ether ketone, ether imide, ether sulfone, phthalamide, phenylene ether, phenylene oxide, phenylene sulfide, sulfone, urethane, vinylidene fluoride and tetrafluoroethylene.

9. The skin whitening composition of claim 3, wherein the silica bead has an average particle size of 100 nm to 1 mm.

10. The skin whitening composition of claim 1, wherein the carbodiimide-based compound is contained at 0.001 to 10 parts by weight with respect to 100 parts by weight of the total composition.

11. The skin whitening composition of claim 1, wherein the reaction accelerating additive, which forms peptide bonds, is contained at 0.0001 to 10 parts by weight with respect to 100 parts by weight of the total composition.

12. The skin whitening composition of claim 1, wherein the skin whitening component contains a carboxyl or amine group in the molecule.

13. The skin whitening composition of claim 1, wherein the skin whitening component is one or more selected from the group consisting of a vitamin and a derivative thereof; flavonoid compounds; resveratrol compounds and a derivative thereof; curcubitacin derivatives; amino acid-based compounds; arbutin, kojic acid, azelaic acid, alpha hydroxy acid, alpha-bisabolol, adenosine, ascorbyl glucoside, ascorbyl tetraisopalmitate, ethylascorbyl ether, fullerene, acetyl tyrosine, diacetyl benzoyl lathyrol, Atractylodes japonica root oil, magnesium ascorbyl phosphate, diosmethine, macelignan, acetyl sphingosine, dihydroxymethoxychalcone, alpha-bisabolol, protocatechuicaldehyde, oryzanol, a licorice extract, a paper mulberry extract, a green tea extract, a morus bark extract, an adlay extract, a yeast extract, an Atractylodes japonica extract, a wheat germ extract, a Paeonia lactiflora extract, a Lithospermum erythrorhizon root extract, a ginseng extract, a red ginseng extract, a Saururus chinensis extract, an Agrimonia eupatoria extract, an Canna indica root extract, a Sasa quelpaertensis extract, a Sophora flavescens root extract, a Coix lacryma-jobi ma-yuen seed extract, a Prunus domestica fruit extract, chitosan, polylysine, glucosamine and a derivative thereof.

14. The skin whitening composition of claim 1, wherein the skin whitening component has a functional group which makes a covalent bond with a protein residue on a skin surface.

15. The skin whitening composition of claim 14, wherein the functional group making a covalent bond with the protein is one or more selected from the group consisting of carbodiimide, imidoester, arylazide, diazirine, hydroxymethyl phosphine, pentafluorophenylester, pyridyl disulfide, sulfo-hydroxysuccinimide ester, alkoxyamine, hydrazide, haloacetyl, azide, carbonate, aldehyde, propionaldehyde, butylaldehyde, nitrophenyl carbonate, aziridine, isocyanate, thiocyanate, epoxide, trecylate, succinimide, hydroxysuccinimidyl ester, imidazole, oxycarbonyl imidazole, imine, thiol, maleimide, vinylsulfone, ethyleneimide, thioether, acrylonitrile, acrylic or methacrylic ester, disulfide, ketone, carboxylic acids and phosphate.

16. The skin whitening composition of claim 1, wherein the skin whitening component is contained at 0.001 to 50 parts by weight with respect to 100 parts by weight of the total composition.

17. The skin whitening composition of claim 1, wherein the carbodiimide-based compound, the reaction accelerating additive and the skin whitening are formulated as an encapsulated single agent, or the carbodiimide-based compound and the reaction accelerating additive; and the skin whitening may be formulated as two separated agents.
